(19) United States
(12) Patent Application Publication
(10) Pub. No.: US 2003/0166149 A1
(43) Pub. Date: Sep. 4, 2003

(54) Method for production of hydroxylated collagen-like compounds

(76) Inventors: Eric Christiaan De Bruin, Wageningen (NL); Marc Willem Theodoor Werten, Wageningen (NL); Frederik Anton De Wolf, Bunnik (NL)

Correspondence Address: YOUNG & THOMPSON, 745 SOUTH 23RD STREET 2ND FLOOR, ARLINGTON, VA, 22202

(21) Appl. No.: 10/340,780

(22) Filed: Jan. 13, 2003

Related U.S. Application Data
(63) Continuation of application No. PCT/NL01/00527, filed on Jul. 11, 2001.

(30) Foreign Application Priority Data
Jul. 11, 2000 (EP) 00202463.6

(51) Int. Cl. C12P 21/02; C07H 21/04; C12N 1/18; C12N 15/74

(52) U.S. Cl. 435/69.1; 435/254.2; 435/320.1; 530/356; 536/23.5

(57) ABSTRACT

The present invention relates to a method for the production of collagen-like compounds containing hydroxylated proline residues. Of specific interest is the production of recombinant collagen-like compounds in which hydroxylation of proline residues is achieved by a prolyl hydroxylase from a fungus, preferably a yeast, in particular Hansenula polymorpha. Also the invention concerns a method for controlling the hydroxylation of proline residues by such a prolyl hydroxylase characterised by the addition of collagen-like oligopeptides, such as gelatine hydrolysate, in particular gelatone or peptone.

### FIELD OF THE INVENTION

The present invention relates to a method for the production of collagen-like compounds containing hydroxylated proline residues.

### BACKGROUND OF THE INVENTION

Collagen is the collective name for a family of fibrous proteins. Structurally, collagen is characterised as being an assembly of three polypeptide chains containing in their primary sequence repeats of -(Gly-Xaa-Yaa)ₙ-triplets which allow for the formation of triple helical domains. In the biosynthesis the polypeptide chains that form collagen undergo several post-translational modifications. Probably the most prominent of these modifications is hydroxylation of proline residues in the Yaa position of the repeating -(Gly-Xaa-Yaa),-triplets to 4-hydroxyproline. It is requisite an appropriate number of proline residues in the Yaa-position is hydroxylated for the protein chains to fold into a triple helical conformation at 37° C. and even at 4° C. non-hydroxylated gelatine does not form triple helices in vitro. If there is no hydroxylation, the peptide chains remain non-helical and cannot self-assemble into stable collagen structures. The enzyme responsible for the hydroxylation of proline in the Yaa position in -Gly-Xaa-Yaa-triplets to 4-hydroxyproline is prolyl 4-hydroxylase.

Collagen is used as a biomaterial in numerous medical applications, such as cosmetic surgery, tissue engineering and wound treatment. Gelatine is denatured and partly degraded collagen. It is also used in various medical and pharmaceutical applications such as capsules, surgical sponges, wound treatment, vaccines, drug delivery systems, it is used in food industry as a gelling agent and it is used in the photographic industry. The most prominent source for natural collagen (and gelatine) is animal bone and hide. However, since long it has been recognised that, in particular for the high-grade medical applications but also for applications requiring a constant composition of collagen or gelatine, alternative sources are desired. In particular production of collagen by micro-organisms would be advantageous to alleviate the immunological, viral- and prion-related hazards that are associated with the use of animal or even human sources of collagen, especially when such collagen is taken up in some form by human subjects. In general, suitable eukaryotic micro-organisms for the production of collagens are fungi and in particular yeasts. It is common knowledge that lower eukaryotic organisms do not possess the post-translational machinery to convert unfolded single chain non-hydroxylated precursor collagens to hydroxylated triple helical collagens.

In particular state of the art is that fungi and in particular yeasts lack the enzyme prolyl 4-hydroxylase. In order to have lower micro-organisms, such as fungi and in particular yeast, producing hydroxylated triple-helix collagen prolyl 4-hydroxylase from animal (human) origin is co-expressed in the microbial host.

Several documents describe the production of hydroxylated collagen in yeast co-expressing prolyl hydroxylase.

In WO93/07889 the synthesis of procollagen or collagen in a variety of cells, including yeast cells, using recombinant DNA systems is described. Animal cells that naturally express prolyl 4-hydroxylase are used. Cells lacking post-translational enzymes may be transformed with genes coding for such enzymes such as prolyl 4-hydroxylase; In the examples is described how Saccharomyces cerevisiae and Pichia pastoris are transformed with recombinant (heterologous) collagen genes and recombinant (heterologous) genes for prolyl 4-hydroxylase.

WO97/14431 is concerned with the production of recombinant procollagen (non-hydroxylated collagen) in yeast. In this document it is explicitly stated that “Yeast does not synthesise the enzyme necessary to hydroxylate proline residues of procollagens”. It is shown that after the introduction of chicken prolyl 4-hydroxylase into the yeast strains GY5196 and GY5198 triple helical structures, stable up to 35° C., were produced. Analysis of collagen triple helix structures gave the direct evidence for the presence of hydroxyproline.

In WO97/38710 the production of collagen in a host cell is described in which a first expression vector comprising a sequence encoding a collagen, and a second expression vector comprising a sequence encoding a post-translational enzyme or subunit thereof are introduced. A variety of host cells, including yeast cells, such as Saccharomyces cerevisiae, Pichia pastoris and Hansenula polymorpha, are mentioned. In the examples the construction of recombinant vectors containing genes for human prolyl 4-hydroxylase and genes for human collagen type III for expression in Saccharomyces cerevisiae and Pichia pastoris is described. The document shows the expression of both human prolyl 4-hydroxylase and human collagen III in a triple helix form in Pichia pastoris.

Also in recent scientific publications the necessity of having the post translational enzyme prolyl 4-hydroxylase co-expressed in yeast has been exemplified. In Vuorela et al. (1997) EMBO J. 16, 6702-6712 and Vaughan et al. (1998) DNA Cell Biol. 17, 511-518 first the yeast Pichia pastoris was engineered to express prolyl hydroxylase and subsequently was shown to produce, upon introduction of the gene for type III procollagen, hydroxylated functional triple helical procollagen III. Toman et al. (2000) J. Biol. Chem, vol. 275, published May 8 (wwwjbc.org, M002284200) describe the production of recombinant human type I procollagen, of which 82% of the proline content compared to tissue derived type I collagen is hydroxylated upon the co-expression of chicken prolyl 4-hydroxylase, resulting in stable triple helix structures.

Several disadvantages are related to the introduction in yeast of prolyl 4-hydroxylase foreign to the yeast for the production of hydroxylated collagens. Extra steps have to be carried out for the construction of appropriate gene constructs containing the information for the expression of prolyl 4-hydroxylase. Introduction of additional gene constructs in yeast cells puts a higher strain on the yeast, resulting in decreasing efficiencies of transformation, thus requiring more material for successful transformation of the yeast. Co-expression of animal (human) hydroxylase results in relatively low yields of collagen (and gelatine) produced by the yeast (Vuorela et al. (1997) EMBO J. 16, 6702-6712 and Keizer-Gunnik et al. (2000) Matrix Biology 19, 29-36). Besides resulting in a low gelatine yield, over-expression of recombinant human prolyl 4-hydroxylase in for instance Pichia pastoris causes changes in cell morphology and inhibition of growth.

WO96/39529 concerns the secretion of heterologous proteins from host cells to which end a mammalian (human) preprocollagen signal is operatively linked to a heterologous protein of interest. Amongst others Hansenula polymorpha is mentioned as host cell. In passing, this document mentions the production of endogenous collagen-like compounds in H. polymorpha. On the basis of 2 terminal sequences of 14 and of 13 amino acid residues respectively, obtained from proteins in the H. polymorpha supernatant, the authors concluded that the proteins were homologous to collagen-related proteins. The two sequences of 14 and of 13 amino acid residues are reported as Gly-Pro-Pro repeats (see sequence listing SEQ ID NO: 8 and SEQ ID NO 9 in WO96/39529). It is mentioned that in matching studies of the found sequences with known collagen sequences the known compounds contain a hydoxylated proline. WO96/39529 does not explicitly describe the presence of hydroxyproline in collagen-like proteins secreted by H. polymorpha, nor does it suggest any sort of applicability from the observation that H. polymorpha secretes collagen-like compounds.

### SUMMARY OF THE INVENTION

It is an object of the present invention to provide a method for the production of collagen and collagen-like compounds comprising hydroxylated proline residues.

The inventors have found that certain fungi, in particular uni-cellular fungi, display prolyl hydroxylase activity contrary to the general notion in the state of the art, teaching that fungi, in particular yeasts, do not possess an enzyme for the post-translational hydroxylation of proline residues in precursor collagens. The art teaches that it is requisite to co-express animal (human) prolyl 4-hydroxylase in order to obtain hydroxylated collagen. The inventors have transformed Hansenula polymorpha in order to produce recombinant collagen without co-expressing any heterologous (exogenous) prolyl 4-hydroxylase. This endogenous hydroxylase activity fundamentally differs from the proline hydroxylase activity observed in some prokaryotic and other organisms, and which is only able to act on free proline, and not on proline residues that are incorporated in a peptide (Shibasaki et al., (1999) Tetrahedron Letters 40, 5227-5230).

### DETAILED DESCRIPTION OF THE INVENTION

Surprisingly, hydroxylated collagen was obtained upon transformation of H. polymorpha only with an expression system comprising a mouse type I collagen sequence and in the absence of any form of information for the expression of exogenous, animal (human), prolyl 4-hydroxylase. Thus, H. polymorpha unexpectedly displays endogenous prolyl hydroxylase activity. Although the experiments have been carried out with the yeast H. polymorpha, it is envisaged that the invention is also applicable to other yeasts and to moulds. Therefore in this specification the term ‘fungus’ or ‘fungal’ is used which covers yeast as well as moulds

In addition to the fungal prolyl hydroxylase activity the inventors have found that this activity can be controlled. Depending on (components in) the cultivation or fermentation medium that is used for growing of the micro-organisms, hydoxylation of proline residues can be induced or prevented. The presence in the medium of enzymatic (tryptic) hydrolysates from animal tissue (e.g. pancreas) results in fungal hydroxylation of proline residues in collagen-like compounds. Although certain co-factors present in the hydrolysate may play a role in the fungal prolyl hydroxylase activity, a principal role must be ascribed to oligopeptides resulting from the hydrolysis of gelatine or collagen like compounds. Probably intact gelatine or collagen-like compounds could act as inducer for prolyl hydroxylation as well, but non-gelling fractions of such compounds are more suited for practical-purposes. Relatively pure gelatine hydrolysate is commercially available under the name gelatone whereas the animal tissue hydrolysate is commercially available as peptone. Omitting gelatine hydrolysate and/or animal tissue hydrolysate from the cultivation or fermentation medium for the micro-organisms prevents fungal hydroxylation of proline residues from occuring.

The examples show that growing H. polymorpha in the presence of peptone results in hydroxylated collagen-like product. Fermentation in the presence of mineral/minimal medium without any supplement or supplemented with casamino acids (casamino hydrolysate) does not result in hydroxylation. The difference in hydroxylase activity is most probably the result of a stimulating effect of peptone. In analogy to various animal cells, collagen receptors at the cell surface could be involved. In this respect a specific, partial, amino acid sequence could play a role.

By nature collagens have a relatively high isoelectric point (pI) of approximately 9.5. Consequently, if carefully isolated and digested, hydrolysates also have a comparable relatively high isoelectric point. The peptone used in the examples has a pI of approximately 9.5. In a comparative experiment a <10 kDa fraction of a tryptic digest of pure gelatine was used which has a pI of approximately 4.5. Comparison of the degree of hydroxylation induced by peptone and induced by the <10 kDa gelatine fraction showed an on average 5 times higher degree of hydroxyalation induced by peptone. It is advantageous therefore to use a collagen like protein which has a relatively high isoelectric point as inducer. It is likely there is an optimum for isolelectric point.

It is envisaged that other proteins than collagen-like proteins may have an inductive effect on hydroxylase activity as well. Preferably such a protein comprises hydroxyproline residues preferably in combination with a high isoelctric point. For instance extensins combine these properties. Extensins play a role in growth, regulation, stress response, cell-cell recognition, and reproductive physiology of plants and are widely distributed throughout the plant kingdom. Extensins are hydroxyproline rich glycoproteins which are also rich in basic amino acids serine, valine, tyrosine, lysine, and in some instances threonine. The polypeptide backbone comprises repeating hydroxyproline. In nature the hydroxyproline component is heavily glycosylated.

Besides gelatine or collagen-like oligopeptides, one or more components, possibly in combination, in peptone could act as (a) cofactor(s) for the hydroxylase in H. polymorpha. Known co-factors for animal prolyl-hydroxylases are ascorbic acid, α-ketoglutarate and Fe²⁺.

Thus, the present invention provides a method for the production of collagen-like compounds containing hydroxylated proline residues characterised by using a fungal prolyl hydroxylase. Preferably the prolyl hydroxylase is from a uni-cellular fungus, preferably from a yeast, in particular from Hansenula polymorpha.

Also a method is provided for the production of collagen-like compounds containing hydroxylated proline residues in which the hydroxylation of proline residues by the fungal prolyl hydroxylase is controlled by the addition of collagen-like oligopeptides, such as gelatine hydrolysate, in particular gelatone or peptone. Preferably the collagen-like oligopeptides have an isoelectric point of higher than 7, more preferably of higher than 8 even more preferably of higher than 9. Theoretically pI values for poly-lysine or poly-arginine of higher than 12 can be obtained. In nature proteins with a pI value of higher than 11.5 are rarely found. In a further embodiment hydroxylation of proline residues by the fungal prolyl hydroxylase is controlled by the addition of an extensin.

In a preferred embodiment of the invention recombinant collagen-like compounds are produced. Recombinant refers to any genetic manipulation of a host organism, such as the introduction of exogenous (heterologous) genes encoding collagen-like compounds or fungal hydroxylase, but also over-expression of endogenous genes encoding collagen-like compounds or fungal hydroxylase and/or combinations thereof.

Production of recombinant proteins and the construction of suitable expression vectors can be conducted according to methods known per se and can for instance be found in Sambrook et al. Molecular cloning: a laboratory manual, Cold Spring Harbor Laboratory Cold Spring Harbor, N.Y., 1989 and Ausubel et al. Current protocols in Molecular Biology, Greene Publisihing Associates and Wiley Interscience, NY 1989. These methods include in vitro recombinant DNA techniques, synthetic techniques and in vivo recombination/genetic recombination.

Several distinct collagen types have been identified in vertebrates, including bovine, ovine, porcine, chicken and human collagens. A comprehensive review of nineteen known collagens is given in WO97/38710. In the method of the invention an expression vector or multiple expression vectors comprising any nucleic acid sequence or combination of nucleic acid sequences encoding natural collagen can be used. In the expression vector additional information may be incorporated for the production of procollagen. Procollagen refers to collagen having additional C-terminal and/or N-terminal peptides that assist in the assembly into trimer, solubility, purification or other function and at some stage are cleaved by N-proteinase, C-proteinase or other proteins to give collagens. Incorporation of such information in the expression vector allows control over the formation of collagen (highly ordered trimeric structure) or gelatine (non-assembled or randomly assembled structure).

Also the expression vector can be equipped with any of a number of suitable transcription and translation elements, including constitutive and inducible promoters, initiation signals, selection markers, secretion signals.

The method of the invention relates to the production of collagen-like compounds and is not limited to the production of (known) natural collagens. Non-natural sequences encoding proteins comprising Gly-Xaa-Yaa repeats or stretches of Gly-Xaa-Yaa repeats are also subject to hydroxylation by fungal hydroxylase. Collagen-like compounds refers to natural and non-natural collagen. The collagen-like compound can be synthetic such as a custom designed amino acid sequence with collagenous, partially non-collagenous or fully non-collagenous nature and/or hydrocolloid, non-hydrocolloid, hydrophilic or hydrophobic nature. Collagen-like compounds according to the invention contain stretches of Gly-Xaa-Yaa triplets, preferably they contain at least 5, more preferably at least 10 consecutive repeats of Gly-Xaa-Yaa triplets. For the collagenous properties of the collagen-like compounds to come about the stretches of at least 5, preferably at least 10 Gly-Xaa-Yaa triplets have to be rich in proline and hydroxy-proline. Although variation occurs, in natural mammalian collagen approximately 20% of the total number of amino acids, including Gly, in stretches of Gly-Xaa-Yaa triplets is proline and/or hydroxyproline. Commonly hydroxyproline is found in the Yaa position. In fish, in particular in cold-water fish, this percentage of proline and/or hydroxyproline is considerably lower, e.g. lower than 15% or even lower than 10%. In non-natural, synthetic or custom-designed collagen-like compounds any percentage of proline residues can be introduced. Thus, collagen-like compounds according to the invention contain stretches of at least 5 preferably at least 10 consecutive repeats of Gly-Xaa-Yaa triplets and at least 5%, preferably at least 10%, more preferably at least 15% of the triplets contain a proline and/or hydroxyproline residue.

Once the prolyl hydroxylase activity has been ascertained in H. polymorpha the enzyme responsible for this activity can be isolated. Different yeast extracts can be fractionated to narrow down the possible protein population(s) giving rise to the hydroxylation activity, eventually the specific protein can be isolated. More specific, the enzyme can be purified and/or isolated by applying column chromatography, in particular affinity chromatography. For example purification and/or isolation of yeast prolyl 4-hydroxylase can be performed by applying a cell lysate of H. polymorpha, grown in medium containing peptone, on a poly L-proline/GPP affinity column (K. I. Kivirikko and R. Myllylä, Methods Enzymol. 1987). As éluent poly L-proline/GPP (3 mg/ml) is suited. Subsequently, a gel filtration step is performed, for example using Superdex 200.

Prolyl 4-hydroxylase activity can be monitored by an in vitro assay based on the hydroxylation coupled decarboxylation of 2-oxo [1-¹⁴C] gluterate (K. I. Kivirikko and R. Myllylä, Methods Enzymol. 1982). Mass finger printing can be performed on the purified enzyme.

Thus the invention also relates to prolyl 4-hydroxylase from a fungus, preferably from Hansenula polymorpha.

The sequence of isolated fungal prolyl 4-hydroxylase can be determined using standard methodology. Based on the sequence of isolated fungal prolyl 4-hydroxylase the genetic information encoding this enzyme can be identified. More specific, the N-terminal amino acid sequence of the H. polymorpha prolyl 4-hydroxylase together with one or more internal amino acid sequences is determined. Specific primers, including degenerate primers, can be designed which, using standard methodology, can be applied to identify, isolate and multiply the prolyl 4-hydroxylase gene. For example by using designed (degenerate) primers the gene, which can be used for further cloning, is isolated by (RT)PCR.

Based on homology with known hydroxylases (degenerate) stretches of oligo-nucleotides can be designed and used to hybridise with yeast DNA. For this purpose particularly useful could be the gelatine or collagen-like compound binding domain of the prolyl hydroxylase enzym. More specific, isolation of the gene can be performed by low stringency oligonucleotide hybridisation on genomic DNA or mRNA isolated from H. polymorpha, with probes based on the alpha subunit of animal prolyl 4-hydroxylases. Finally the gene encoding the prolyl 4-hydroxylase can be isolated using degenerate primers based on the catalytically important alpha subunit sequences of animal and viral hydroxylases.

Based on the gene for prolyl 4-hydroxylase isolated from H. polymorpha it is possible to identify related genes in other fungi, in partciular in other yeast strains. The related genes in other yeast strains will probably result in similar proteins having similar activity like prolyl 4-hydroxylase from H. polymorpha.

The isolated nucleotide sequence encoding fungal, in particular yeast, prolyl 4-hydroxylase can be incorporated in expression vectors and used to transform microbial hosts. The expression vector may also comprise animal (human) collagen genes. In particular fungi, especially uni-cellular fungi or fungus-like eukaryotic microorganisms can be transformed in order to express fungal, in particular yeast, prolyl hydroxylase, preferably in combination with the expression of recombinant collagen. Particularly preferred are yeast cells for the expression of yeast prolyl 4-hydoxylase, preferably in combination with the expression of recombinant collagen in order to produce hydroxylated collagen.

Another aspect of the invention concerns the production of fungal endogenous (homologous) collagen-like compounds. Polypeptides comprising stretches of Gly-Xaa-Yaa triplets, more specific, stretches of Gly-Pro-Pro triplets can be identified in and isolated from yeast, in particular Hansenula polymorpha. Upon the action of yeast (Hansenula polymorpha) prolyl 4-hydroxylase this collagen-like compound is hydroxylated. Specifically the proline residue in the Yaa position is hydroxylated to 4-hydroxy proline. Such microbial (endogenous yeast) collagen-like compounds can be an alternative for animal or human collagen. The natural non-animal proteins are free of prions and viruses. The endogenous hydroxylase activity in yeast does not inhibit growth and does not decrease the yield of collagen-like compound in H. polymorpha. Other fungi can possess (part of) the genes and machinery for expression of endogenous (hydroxylated) collagen-like compounds. Therefore other expression hosts, such as fungi and in particular yeasts, can be used for the production of endogenous, H. polymorpha, collagen-like compounds.

In this aspect of the invention a method is provided for the production of endogenous fungal collagen-like compounds comprising the steps of culturing a fungus or fungus-like eukaryotic microorganism, and isolating the endogenous fungal collagen-like compound. Preferably the fungus is a uni-cellular fungus, preferably a yeast, in particular Hansenula polymorpha. Preferably the endogenous yeast collagen-like compound is from H. polymorpha.

According to the invention H. polymorpha prolyl 4-hydroxylase can be (over)expressed in H. polymorpha or in other microbial hosts for the production of hydroxylated collagen-like compounds. The collagen-like compounds can be exogenous (heterologous) or endogenous (homologous) to the microbial host.

By proteomic tools the gene encoding the collagen-like protein of H. polymorpha can be isolated. After in-gel tryptic digestion of the protein, internal amino acid sequences can be determined by Q-tof analysis. Subsequently, by degenerate primer design and (RT)PCR the gene encoding the collagen-like protein can be isolated.

Also according to the invention H. polymorpha gene(s) encoding collagen-like compounds can be (over)expressed in H. polymorpha or in other microbial hosts, preferably in combination with fungal prolyl 4-hydroxylase, for the production of (hydroxylated) collagen-like compounds.

Preferred hosts for the production according to the invention are fungi or fungus-like eukaryotic microorganisms. Suitable moulds are of the genera Aspergillus, Rhizopus and Trichoderma. In particular useful systems for the production of hydroxylated collagen or collagen like compounds are uni-cellular fungi, in particular yeast cells. Preferred industrially applicable yeast cells for the production of proteins on a commercial scale are Hansenula polymorpha, Pichia pastoris, Saccaromyces cerevisiae, Kluyveromyces lactis, Yarrowia lypolitica and Cryptococcus curvatus, but other microbial hosts may prove to be applicable as well.

A particularly useful micro-organism is the methylotrophic yeast Hansenula polymorpha. Growth on methanol results in the induction of key enzymes of the methanol metabolism such as MOX, DAS and FMDH, which can constitute up to 30-40% of the total cell protein. The genes encoding MOX, DAS and FMDH production are controlled by very strong inducible promoters. Any single one or combination of two or all three of these promoters can be used to obtain high level expression of heterologous genes in H. polymorpha. Genes encoding collagens and/or fungal prolyl hydroxylase of interest are cloned into an expression vector under the control of an inducible H. polymorpha promoter. If secretion of the product is desired, a polynucleotide encoding a signal sequence for secretion in yeast, such as the S. cerevisiae prepro-mating factor α1 is fused in frame with the coding sequence for the collagen and/or fungal prolyl hydroxylase of interest. Additionally the expression vector may contain an auxotrophic marker such as URA3 or LEU2.

By applying known techniques the expression vector is used to transform H. polymorpha host cells. A particular useful feature of H. polymorpha is the spontaneous integration of of up to 100 copies of the expression vector into the genome. Mostly the integrated DNA forms multimers exhibiting head to tail arrangement. Integrated foreign DNA has been shown to be mitotically stable in several recombinant strains, even under non-selective conditions. The phenomenon of high copy integration further adds to the high productivity potential of the system.

As is described hereinabove the hydroxylase activity can be controlled by the addition of a suitable inducer to the culture or fermentation medium of the host organisms. As is mentioned a suitable inducer is a collagen-like oligopeptide. Advantageously such a suitable inducer does necessarily have to be a collagen-like oligopeptide of animal origin such as peptone which is prominently used in the examples. A suitable inducer could also be (1) produced recombinantly in microbial or plant systems, (2) an endogenous yeast collagen-like protein from H. polymorpha as described hereinabove, or (3) chemically synthesized. Thus, a particular advantage of the invention is that a completely animal-free recombinant collagen- or gelatine production system is obtained.

### BRIEF DESCRIPTION OF THE FIGURES

FIG. 1: Constitutive expression of recombinant gelatine by H. polymorpha. An arrow indicates the 15 kDa gelatine product.

FIG. 2: Methanol induced expression of recombinant gelatine by H. polymorpha. An arrow indicates the 15 kDa gelatine product.

FIG. 3: SDS-PAGE, stained with Coomassie Brilliant Blue, of proteins from H. polymorpha cells grown in YPD medium. M: Low molecular weight protein marker (Pharmacia). Lane 1, Supernatant after heat treatment at 70° C. and removal of cells; Lane 2, Protein precipitated at 40% (vol.) acetone; Lane 3, Protein precipitated after removing the 40% precipitate and bringing the 40% (vol.) acetone supernatant to 80% (vol.) acetone. An arrow indicates the 38 kDa collagen-like protein

FIG. 4: SDS-PAGE of the YPD medium in which H. polymorpha cells were grown. M: Low molecular weight protein marker (Pharmacia). Lane 1, YPD medium after removal of cells; Lane 2, 40% (vol.) acetone precipitate of the medium; Lane 3, Protein precipitated after removing the 40% precipitate and bringing the 40% (vol.) acetone supernatant to 80% (vol.) acetone.

FIG. 5: SDS-PAGE of extracellular proteins produced by H. polymorpha during glucose fed-batch fermentation on medium supplemented with peptone. 10 μL of culture supernatant was loaded in each well. Lane 1, 2, 3; After 24, 40 and 70 hours of fermentation, respectively. M: Broad range precision protein standards (Bio-Rad). A 38 kDa enedogenous protein band is observed.

### EXAMPLES

### Example 1

Materials and Methods

Yeast Strain and Plasmids

The H. polymorpha strain NCYC 495 leu1.1, which is deficient in beta-isopropylmalate dehydrogenase (LEU 2) was used for recombinant gelatine production. For methanol induced gelatine expression we used the plasmid pHIPX4, which contains a LEU selectable marker, a kanamycin resistance marker and an expression cassette, containing the methanol oxidase (MOX) promoter and the amino oxidase (AMO) terminator. For constitutive gelatine expression we used the plasmid pHIPX7, which is the same as pHIX4 with the exception that the expression cassette, contained the transcription elegation factor (TEF1) promoter instead of the MOX promoter. A 1268 bp HindIII/XhoI fragment, containing the S. cerevisiae α-mating factor prepro signal and 1.0 kb of the helical domain of mouse type I collagen, from the vector pCOL1A1-1, was inserted into the Hind III/Sal I site of the vectors pHIPX4 and pHIPX7. This yielded pHIX4-1A1 and PHIX7-1A1. All molecular techniques were performed as described by Sambrook et al. Molecular cloning: a laboratory manual Cold Spring Harbor Laboratory, Cold Spring Harbor, N.Y., 1989, or according to manufacturers protocols.

Transformation of H. polymorpha

Plasmids used for transformation were linearised with Sca I. Transformation of H. polymopha by electroporation was performed according to Faber et al. (1994) Hansenula polymorpha. Curr. Genet., 25, 305-310, using a GenePulser (Bio-Rad). After growth on minimal glucose plates at 37° C. for 3 days, several colonies were selected for PCR confirmation. Cells were used directly for PCR without any pretreatment using the 1A1-1 FW-primer (SEQ ID NO: 1): 5′-CTTCCCAGATGTCCTATGGCTATGATG-3′ and the AMO-primer(SEQ ID NO: 2): 5′- TGTCCTTGGTCTCCTTGTGCACG-3′.

Media Compositions

Minimal glucose plates, for selection of transformants, contained 1.34% yeast nitrogen base without amino acids (Difco), 1% glucose and 1.5% agar. Mineral glucose medium was used to preculture H. polymorpha for fed-batch fermentation expression experiments and contained per litre: 2.5 g ammonium sulfate, 0.25 g magnesium sulfate heptahydrate, 0.7 g di-potassium hydrogen phosphate trihydrate, 3.0 g sodium dihydrogen phosphate monohydrate, 0.5 g yeast extract, 50 g glucose, 0.02 mg biotin, 0.6 mg thiamin and 1 mL of Vishniac trace elements solution.

Fermentation basal salts medium contained per litre: 26.7 ml phosphoric acid (85%), 0.93 g calcium sulfate dihydrate, 18.2 g potassium sulfate, 14.9 g magnesium sulfate heptahydrate, 4.13 g potassium hydroxide and 4.3 ml of trace elements. Trace salts solution contained per litre: 4.5 g cupric chloride dihydrate, 0.09 g potassium iodide, 3.5 g manganese chloride tetrahydrate, 0.2 g sodium molybdate dihydrate, 0.02 g boric acid, 1.08 g cobalt sulfate heptahydrate, 42.3 g zinc sulfate heptahydrate, 65.0 g ferrous sulfate heptahydrate, 0.6 g thiamine, 0.2 g biotin and 5.0 ml sulfuric acid (96%).

Fermentative Production of Gelatine by H. polymorpha

Fed-batch fermentations of H. polymorpha transformants were performed in a 1 L fermenter (Applikon). At the start of the fermentation, the fermenter contained 450 ml FBS medium, to which, when indicated 50 ml of a 10% (w/v) casamino acids solution (Merck), 50 ml of a 10% (w/v) peptone solution (Duchefa) or 50 ml of an ultrafiltrated 10% (w/v) peptone solution was added.

60 g/l glucose (w/v) was used as carbon-source during batch phase. The temperature was set at 37° C., the agitation at 500 rpm and the aeration rate at 1 L/min. For optimal result the pH was adjusted to pH 5.0 with ammonium hydroxide (25%). The fermenters were inoculated with a pre-culture of 50 mL. When the glucose of the batch phase was completely consumed, the aeration and the agitation were increased to 2 L/min and 1000 rpm, respectively. The fed-batch phase was initiated by feeding a 50% glucose (w/v) solution, containing 12 ml/L trace salts, at a rate of 10 mL/h. The pH was maintained at 5.0 by the addition of 25% ammonium hydroxide.

An additional 5 g of casein hydrolysate or 5 g of peptone were supplemented to the medium when wet cell weight reached about 180 g/l. For the constitutive gelatine expression by PHIX7-1A1 transformants the glucose fed-batch phase was then continued. For methanol induced expression of gelatine by pHIX4-1A1 transformants a methanol fed-batch was initiated by feeding 100% methanol, containing 12 ml/l trace salts. The feed rate was initially set at 1 ml/h and was gradually increased to maximally 8 ml/h. During fed-batch phases the dissolved oxygen concentration was kept above 20% to avoid oxygen limitation. Throughout the fermentations 2 ml culture samples were taken at intervals of about 12 hours. Samples were spun at 20,000 g for 1 min and the supernatants were filtered using disposable 0.22 μm filters.

Sodium Dodecyl Sulfate (SDS)-Polyacrylamide Gel Electrophoresis (PAGE), N-Terminal Protein Sequencing and Immunoblotting

SDS-PAGE was performed in a Mini PROTEAN II system (Bio-Rad) under reducing denaturing conditions. Gels were stained with Coomassie Brilliant Blue (CBB R-350) For N-terminal protein sequencing, protein was blotted onto Immobilon P^{SQ} (Millipore) by applying 100V for one hour in a Mini Trans-Blot Cell (Bio-Rad). Transfer buffer was 2.2 g CAPS per liter of 10% methanol, pH 11. Blots were stained with Coomassie Brilliant Blue (CBB R-350) and selected bands were cut out. N-terminal sequencing using Edman-degradation was performed.

For immunoblotting, protein was electrophorectically transfered onto a PVDF filter, and the filter was blocked with 5% skim milk powder in TBST (0.1 M Tris-HCl, pH 7.5; 1.5 M NaCl; 0.1% Tween-20) at room temperature for 1 h. The filter was incubated overnight with monoclonal anti-myc antibody (Roche; 1: 20.000 in 1% skim milk in TBST), washed with TBST, and incubated for 1 h with a secondary antibody-conjugated to alkaline phosphatase (AP) (goat anti-mouse, Sigma; ; 1: 10.000 in 1% skim milk in TBST). The filter was washed with TBST and then rinsed with AP buffer (0.1 M Tris-HCl, pH 9.5; 0.5 M MgCl₂; and 0.1 M NaCl). Antibody-binding was detected by incubating the filter in 10 ml AP buffer containing 33 μl of 5-bromo 4-choro 3-indoyl phosphate (50 mg/ml) and 66 μl of nitro-blue tetrazolium (50 mg/ml) (USB).

Degree of Hydroxylation

The degree of hydroxylation of proline residues in the Yaa position of the Gly-Xaa-Yaa triplets in both endogenous (see example 2) collagenous proteins and recombinant proteins was calculated as follows: The development and the decay of glycine, proline and hydroxyproline peaks in successive amino acid sequencing steps was analyzed by comparing the relative signal intensities of each amino acid obtained in successive steps.

First the decay rates were analyzed in steps that by itself did not give rise to a new signal of the same amino acid, e.g. steps 4, 5, 7, 8 and 10 for hydroxyproline. The decay rates were then interpolated for sequencing steps that gave rise to new proline- or hydroxyproline signals and the proline or hydroxyproline signals remaining from previous steps were subtracted from the new signal in order to evaluate the additional signal (corrected signal) obtained in each step. The signals were also corrected for the slow overall decay of sensitivity observed for successive triplets. Finally, the sum of the corrected proline and hydroxyproline signals in sequence steps 3, 6, and 9 were compared with the corrected proline signals in steps 2, 5 and 8, assuming that the corrected signals in successive steps correspond to approximately equimolar amounts of material:

Pᵢ₋₁=Pᵢ+C.Oᵢ

Here, P and O are the corrected proline and hydroxyproline peak heights, respectively, i is the sequencing step number and C is an unknown conversion factor, relating the relative intensities of the proline and hydroxyproline signals. As C can be calculated from this equation, the degree of hydroxylation of proline residue i (just N-terminal to glycine residue i+1) can be calculated as:

% OHₘ=100*C.Oᵢ/(Pᵢ+C.Oᵢ)

Results

Constitutive and methanol induced production of rec. hydroxylated gelatines A 1268 bp HindIII/XhoI DNA fragment from vector pCOL1A1-1, containing the S. cerevisiae α-mating factor prepro signal fused to the 1.0 kb mouse COL1A1 cDNA fragment encoding a gelatine molecule with a theoretical molecular weight of 28 kDa, was cloned in H. polymorpha expression vectors pHIPX7 and pHIPX4. The vectors pHIPX7-1A1 and pHIPX4-1A1 thus obtained were used to transform H. polymorpha, so as to allow constitutive and methanol-induced recombinant gelatine expression, respectively.

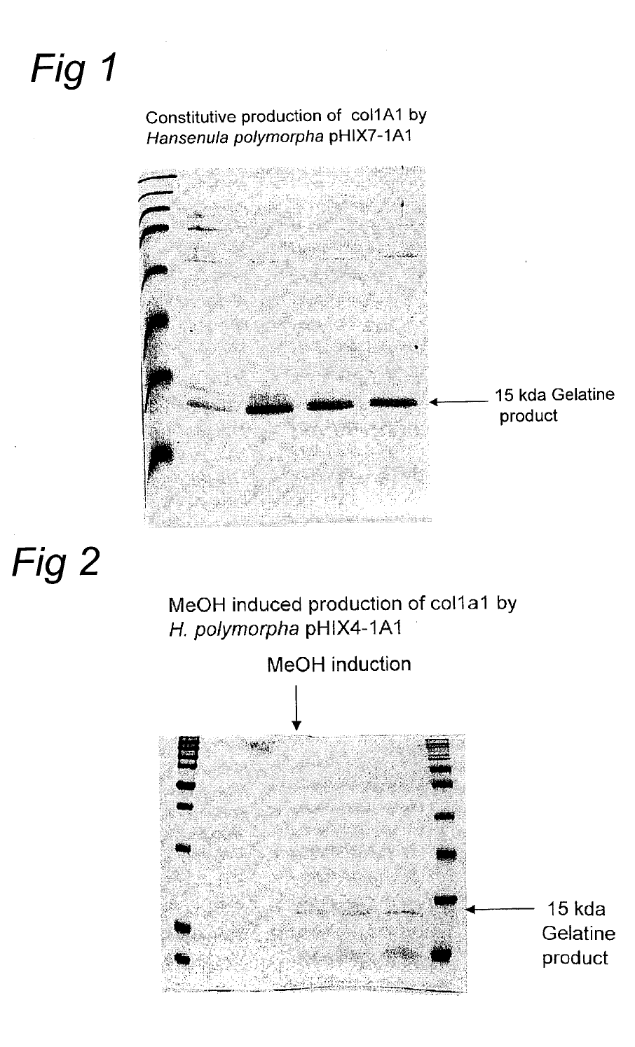

After colony PCR, transformants were selected for fermentation in mineral FBS medium. SDS-PAGE analysis showed the constitutive and methanol-induced production of gelatine in extracellular medium using the pHIPX7 and pHIPX4 transformants, respectively (see FIGS. 1 and 2, respectively). Expression medium, was supplemented with peptone. A degradation product of COL1A1 with an apparent molecular weight of 15 kDa could be observed in all fermentations.

15 Kda gelatine protein bands of the different fermentations were excised from the blots and N-terminal amino acid sequences were determined.

N-terminal aminoacid sequences of produced gelatine produced during different fermentation are given in the following table 1.

The N-terminus found is indeed an internal sequence of the recombinant COL1A1 cDNA gene product. Moreover, when peptone was supplemented to the medium prolines in the product were hydroxylated to 4-hydroxyprolines.

To exclude the possibility that the observed 15 kDa collagen fragment was derived from the peptone added to the growth medium, a low molecular weight fraction of peptone was used in a new fermentation, and the recombinant gelatine product was carefully separated from peptone remnants in the medium.: Of a 10% (w/v) peptone solution, 50 ml was ultra-filtered using a 10 kDa cut-off filter. Low molecular weight components and peptides of the peptone, which passed the membrane, were added to the fermentation medium during an expression experiment with the pHIPX4-1A1 transformant. SDS PAGE of the added ultrafiltrated peptone fraction of <10 kDa showed no protein bands higher than 10 kDa (figure not shown). The recombinant gelatine fragment expressed and secreted by the cells was subsequently ultra-filtered with a new 10 kDa cut-off filter of the same type and washed 3 times with destined water to remove residual <10 kDa peptone remnants. SDS PAGE of the ultrafiltrated and subsequently washed fermentation supernatant showed a band at 15 kDa (figure not shown). N-terminal sequencing of the purified 15 kDa product, obtained after SDS-PAGE and blotting, revealed the internal sequence of the recombinant gelatine and the presence of hydroxylated prolines (Table 1).

Due to the different sequence of the recombinant gelatine, as compared to the the poly [Gly-Pro-Pro] stretch of endogenous H. polymorpha protein (see example 2) and due to some noise in sequencing data, the degree of hydroxylation in the Yaa position of the recombinant gelatine was difficult to calculate according to the procedure described in the material and methods section. The estimates of Hyp/(Pro+Hyp) in the Yaa position in various determinations varied from 25 to 50 mol %, with an average value of about 35 mol %.

In order to investigate the specificity of the induction for the supplement added to the growth medium, peptone was compared with: (1) casamino acids, which, like collagen, are rich in proline, (2) free hydroxyproline, (3) a mixture of free amino acids mimicking the overall amino acid composition of peptone, (4) pure gelatine (i.e. deamidated and partially degraded animal type I and III collagen) which was previously digested with trypsin, heat-treated to inactivate trypsin again, and ultrafiltered to remove the >10 kDa fraction, (5) synthetic polyproline, and (6) synthetic poly-4-hydroxyproline. The results are shown in Table 1: only with the hydroxylated gelatine <10 kDa digest in the growth medium, specific peptidyl-prolyl-4-hydroxylation of recombinant gelatine was obtained, during expression in H. polymorpha. As compared to peptone, the resulting level of hydroxylation of recombinant gelatine was low (5-10%).

It is noted that suitable collagen-like inducer peptides need not necessarily be of animal origin, but could be (1) produced recombinantly in microbial or plant systems, (2) endogenous yeast collagen-like proteins such as detected in H. polymorpha (see example 2), or (3) chemically synthesized. Thus, a completely animal-free recombinant collagen- or gelatine production system can be obtained. In analogy to various animal cells, collagen receptors at the cell surface could be involved.

In order to elucidate the active component in peptone involved in the observed proline-hydroxylation activity a composition was prepared containing certain known co-factors for animal prolyl-hydroxylases. The possible presence of these co-factors in peptone might be responsible for activation of hydroxylation enzymes. Fermentation medium was supplemented with, amongst others: ascorbic acid, α-ketoglutarate, Fe²⁺sulphate. This composition was added (two times) to the fermentation medium (mineral/minimal medium) during the expression of recombinant gelatine in H. polymorpha. No hydroxylation of the produced gelatine was observed. Thus, these co-factors are not essential in the hydroxylation of recombinant gelatine in H. polymorpha.

TABLE 1
N-terminal aminoacid sequence of produced gelatine
under various conditions.

| Growth medium supplement | N-terminal amino acid sequence |
|---|---|
| Constitutive expression | |
| — | GFQGPPGEP (SEQ ID NO:3) |
| casamino acids (1%) | GFQGPPGEP (SEQ ID NO:4) |
| peptone (1%) | GFQGPZGEZ (SEQ ID NO:5) |
| free 4-hydroxyproline | GFQGPPGEP (SEQ ID NO:6) |
| MeOH induced expression | |
| casamino-acids (1%) | GFQGPPGEP (SEQ ID NO:7) |
| peptone (1%) | GFQGPZGEZ (SEQ ID NO:8) |
| <10 kDa peptone fraction | GFQGPZGEZ (SEQ ID NO:9) |
| free aminoacids | GFQGPPGEP (SEQ ID NO:10) |
| free 4-hydroxyproline | GFQGPPGEP (SEQ ID NO:11) |
| <10 kDa bovine gelatine tryptic digest | GFQGPZGEZ (SEQ ID NO:12) |
| poly-L-proline | GFQGPPGEP (SEQ ID NO:13) |
| poly-L-4-hydroxyproline | GFQGPPGEP (SEQ ID NO:14) |

Z = 4-hydroxyproline

Conclusion: It is possible to produce hydroxylated recombinant gelatines by H. polymorpha, using no exogenous hydroxylase. The production is independent of the mode of expression, i.e. constitutive or MeOH induced. 4-Hydroxyproline residues are only found in the Yaa position of the triplets. Also it is possible to control the prolyl hydroxylase activity in H. polymorpha by the use of peptone in the fermentation medium.

### Example 2

Abbreviations: CAPS, 3-cyclohexylamino-1-propanesulfonic acid; CBB, Coomassie Brilliant Blue; HPLC, high performance liquid chromatography; Hyp, 4-hydroxyproline; PAGE, polyacrylamide gel electrophoresis; SDS, sodium dodecyl sulfate; vvm, volume (L of air) per volume (L) of fermentation broth per minute; YPD, yeast extract, peptone and dextrose.

Materials and Methods

Yeast Strain

The yeast strain Hansenula polymorpha NCYC 495 was used in all experiments.

Cultivation Medium and Growth Conditions in Shake Flasks

H. polymorpha was grown at 37° C. in YPD medium (1% yeast extract, 2% peptone, and 2% glucose; Duchefa), or in mineral glucose medium, which contained per liter 2.5 g ammonium sulfate, 0.25 g magnesium sulfate heptahydrate, 0.7 g di-potassium hydrogen phosphate trihydrate, 3.0 g sodium dihydrogen phosphate monohydrate, 0.5 g yeast extract, 50 g glucose, 0.02 mg biotin, 0.6 mg thiamin and 1 mL of Vishniac trace elements solution.

Cultivation Medium and Growth Conditions in Fed-Batch Fermentation

Fed batch fermentation of H. polymorpha was performed in a 1 L fermenter (Applikon). At the start of the fermentation, the fermenter contained 500 mL fermentation basal salts medium, to which 5 g of casein hydrolysate (Merck) or 5 g of peptone (Duchefa) were added. Fermentation basal salts medium contained, per liter: 26.7 mL of phosphoric acid (85%), 0.93 g calcium sulfate dihydrate, 18.2 g potassium sulfate, 14.9 g magnesium sulfate heptahydrate, 4.13 g potassium hydroxide and 4.3 mL of trace elements. Trace elements contained per liter: 4.5 g cupric chloride dihydrate, 0.09 g potassium iodide, 3.5 g manganese chloride tetrahydrate, 0.2 g sodium molybdate dihydrate, 0.02 g boric acid, 1.08 g cobalt sulfate heptahydrate, 42.3 g zinc sulfate heptahydrate, 65.0 g ferrous sulfate heptahydrate, 0.6 g thiamine, 0.02 g biotin and 5.0 ml sulfuric acid (96%). Glucose, 60 g/L was used as a carbon-source during batch phase fermentation. The temperature was set at 37° C., the agitation at 500 rpm and the aeration rate at 1 vvm. The pH was adjusted to pH 5.0 with ammonium hydroxide (25%).

The fermenter was inoculated with 50 ml of a culture grown overnight in mineral glucose medium. When the glucose of the batch phase was completely consumed, an additional 5 g of casein hydrolysate, or 5 g of peptone was added to the fermenter. The same type of supplement was consistently used at this stage and at the start of the fermentation. Subsequently, the aeration and the agitation were increased to 2 vvm and 1000 rpm, respectively and the fed-batch phase was initiated by feeding a 50% (w/v) glucose solution, containing 12 mL/L trace salts, at a rate of 10 mL/h. The pH was maintained at 5.0 by the addition of 25% ammonium hydroxide. During the whole fermentation the dissolved oxygen concentration was kept above 20% to avoid oxygen limitation. The fermentation was stopped when the cell wet weight reached about 300 g/L. Throughout the fermentation 2 mL culture samples were taken at intervals of about 12 hours. Samples were spun at 20,000 g in a micro-centrifuge for 1 min and the supernatants were filtered using disposable 0.22 μm filters.

Heat Treatment of H. polymorpha Cells

Heat treatment of H. polymorpha cells was performed as follows: 20 ml cultures of H. polymorpha were grown to an optical density of 1.5 at 600 nm measured in a Corning calorimeter 254, using disposable 10×4×45 mm cuvettes. Cells were harvested by centrifugation at 3,000 g for 10 min, washed four times with 100 mM NaCl, to remove medium components, and resuspended in 0.5 mL of 100 mM NaCl. The cells, in a closed 1.5 mL plastic tube (Eppendorf) were subsequently heat treated for 20 min in a 70° C. water bath, placed on ice for 1 min and centrifuged at 20,000 g in a micro-centrifuge. Microscopic analysis of cells showed that the heat treatment did not cause detectable cell-lysis. The supernatant was analysed for the presence of collagenous proteins.

Differential Acetone Precipitation

Acetone, previously chilled to 0° C., was added dropwise to chilled cell free supernatant of heat-treated H. polymorpha cells. The resulting protein precipitates were centrifuged for 15 min at 20,000 g in a micro-centrifuge.

Hydroxyproline Detection

The amount of protein was first determined using the bicinchoninic acid (BCA) assay purchased from Pierce. Vacuum dried samples of 10 μg protein were hydrolyzed in 6N HCl vapour at 110° C. overnight on a Waters Pico Taq workstation (Waters Corporation). Detection of free hydroxyproline was performed as described by Creemers et al. (1997) BioTechniques 22:656-658.

Total Amino Acid Composition

Protein (10 μg) was hydrolyzed as described for hydroxyproline detection. The free amino acids were 6-aminoquinolyl-N-hydroxysuccinimidyl carbamate-derivatized using the AccQ Taq method (Waters Corporation). Derivatized amino acids were analysed on a Waters 600 S HPLC system equipped with a Jasco 820-FP detector and a Waters Novapak C18 reverse phase column.

SDS-PAGE and N-Terminal Sequencing

Polyacrylamide gelelectrophoresis (PAGE) in the presence of sodium dodecyl sulfate (SDS) as a denaturing agent, was carried out using the buffer-system of Laemmli (1970) Nature, 227:680-685, on the Bio-Rad Mini PROTEAN II system (7 cm×10 cm) under reducing conditions. 15% acrylamide gels of 0.5 mm thickness were used. Gels were stained with 0.1% Coomassie Brilliant Blue (PhastGel Blue R-350, Pharmacia) in 10% MeOH in water containing 10% acetic acid for 1 h and destained by boiling the gel in a magnetron for 10 min in a 1L beaker, containing 700 ml water. For N-terminal protein sequencing, proteins were blotted onto Immobilon P^{SQ} (Millipore) by applying 100 V for one hour in a Mini Trans-Blot Cell (Bio-Rad). Transfer buffer was 2.2 g CAPS per liter of 10% methanol, pH 11. Blots were stained with Coomassie Brilliant Blue (Phastgel Blue R-350) and selected bands were cut out. N-terminal sequencing using Edman-degradation was performed. The amount of 4-hydroxyproline in the sequencing reaction was quantified.

Results

Isolation and Purification of a Collagenous Protein from Shake Flask Cultures

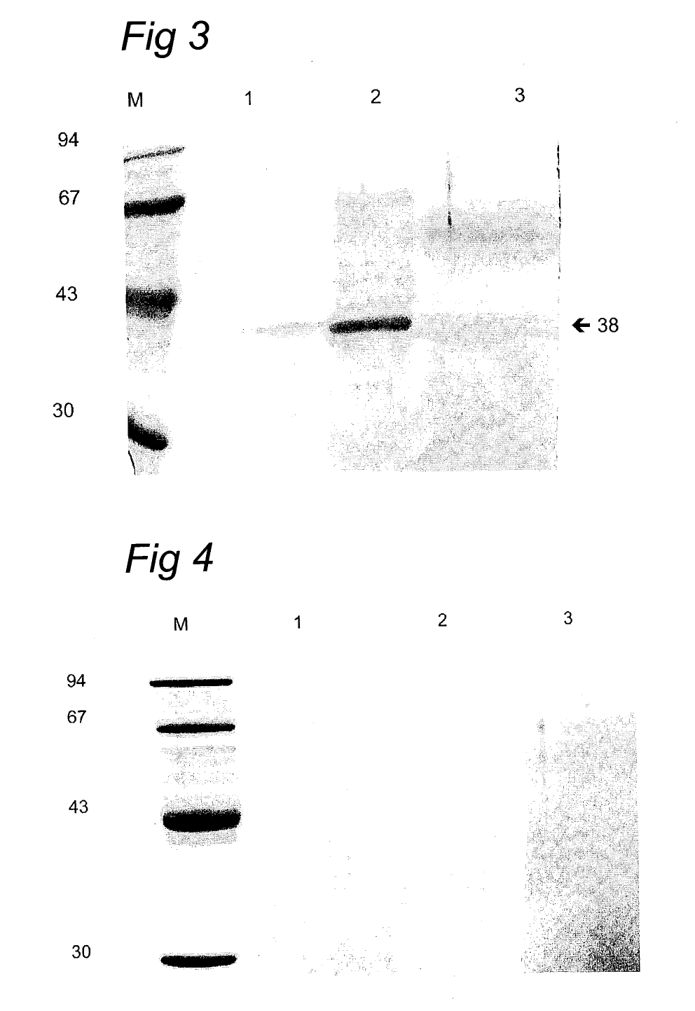

Upon heat treatment of washed H. polymorpha cells grown in YPD medium proteins were released. A 38 kDa protein band was observed, see SDS-PAGE analysis FIG. 3, lane 1. No proteins were released from washed H. polymorpha cells when incubated at 37° C. instead of 70° C. In contrast to the fermentation medium described above, the YPD medium in which the cells were grown in shake flasks did not contain protein bands, as shown by SDS-PAGE analysis (FIG. 4 lane 1). Also this shows that the proteins in FIG. 3 were not derived from the medium, but from the cells themselves. Because microscopic analysis of cells showed that the heat treatment did not cause detectable cell-lysis, the proteins were probably derived from the cell surface. In the fermenter, as opposed to shake flasks, the observed proteins were released from the cells, probably due to shearing forces. In order to investigate the possible collagenous nature of the proteins released from the washed, heat treated cells grown in shake flasks, differential acetone precipitation was performed on the released H. polymorpha proteins. Werten et al. (1999) Yeast 15:1087-1098 used differential acetone precipitation to separate non-collagenous extracellular Pichia pastoris proteins, precipitating at 40 volume % of acetone, from recombinant collagen-like proteins, precipitating at 80 volume % of acetone. Some of the proteins released by heat treatment of washed H. polymorpha cells precipitated at 40 volume % acetone (FIG. 3, lane 2). After removal of the 40% acetone precipitate and increasing the acetone concentration in the supernatant from 40 to 80 volume %, other proteins precipitated (FIG. 3, lane 3). This fraction is referred to as the 40-80% acetone precipitate. Protein precipitates were dissolved in a 5 times less volume of water than the starting volume before acetone precipitation, so acetone precipitation did also have a concentrating effect. The most prominent protein band, the size of about 38 kD, in the SDS-PAGE gel of the 40-80% precipitate, derived from 20 mL shake flask culture, corresponded to about 1 μg protein, as estimated from the intensity of the Coomassie-stained band. Also the YPD medium in which the cells were grown, was differential acetone precipitated but no distinct protein bands were detected (FIG. 4, lane 2 and 3).

The hydroxyproline assay showed that the 40-80% acetone precipitate of the protein, derived from the washed cells by heat treatment, contained 8% (w/w) hydroxyproline, after hydrolysis of the dried protein precipitate. No hydroxyproline was detected in the 40% acetone precipitate of the cell-derived protein. Analysis of the entire amino acid composition further confirmed the collagenous nature of the cell-derived protein in the 40-80% precipitate. High amounts of glycine (26.2 mol %), proline (9.9 mol %) and 4-hydroxyproline (9.8 mol %) were observed. This indicates an overall abundance of collagenous proteins in this fraction.

Subsequently, the N-terminal amino acid sequences of the most abundant proteins in each of the acetone-precipitated fractions were determined, viz. a 40 kD protein in the 40%, and a 38 kD protein in the 40-80% acetone precipitate, as shown in FIG. 3. The results are given in Table 2. The N-terminal sequence of the 40 kD protein in the 40% acetone precipitate (Table 2) was not collagenous, but the N-terminus of the 38 kD protein in the 40-80% acetone precipitate consisted of at least seven successive [Gly-Pro-Hyp] triplets. Possibly there are even more contiguous [Gly-Pro-Hyp] triplets, as sequencing was terminated after 21 amino acids. To our knowledge, such long stretches of contiguous [Gly-Pro-Pro]/[Gly-Pro-Hyp] triplets are not present in any animal collagen.

TABLE 2
N-terminal amino acid sequences of acetone fractionated proteins of
heat treated H. polymorpha cells.

| Protein size (kD) | Precipitate | N-terminal amino acid sequence |
|---|---|---|
| 40 (band fig 3 lane 2) | 40% acetone | EASLGFDLGVQATDGSXKTA |
| | | (SEQ ID NO:15) |
| 38 (band fig 3 lane 3) | 40-80% acetone | GPZGPZGPZGPZGPZGPZGPX |
| | | (SEQ ID NO:16) |

Z = 4-hydroxyproline, X = un-identified amino acid residue

Exclusively the proline residues only in the Yaa position of the [Gly-Xaa-Yaa] were hydroxylated. This strict sequence specificity is the same as observed in animal collagens. The development and the decay of the glycine-, proline-and hydroxyproline peaks in successive amino acid sequencing steps was analyzed by comparing the relative signal intensities in sequencing chromatograms of each amino acid obtained in successive steps. Thus, the degree of hydroxylation in position Yaa of the three most N-terminal [Gly-Xaa-Yaa] triplets was estimated to be in the range of 50-65 mol %. As the prolines in position Xaa were never hydroxylated, the overall level of prolyl hydroxylation in the first three [Gly-Pro-Pro] triplets was 25-28 mol %. Note that in stretches with a low incidence of proline in the Xaa position of the triplets, the average degree of hydroxylation will approach the degree ocurring in the Yaa position, e.g. 50-65 mol %. The amino acid analysis described above indicated an overall degree of prolyl hydroxylation of approximately 50 mol % in the 40-80% acetone precipitate of washed, heat-treated cells.

The 38 kDa protein isolated from a 20 ml shake flask culture with an optical density at 600 nm of 0.100 corresponded to about 0.5 μg of protein (i.e. 25 μg protein released/1 culture at low cell density), as estimated from the intensity of the Coomassie-stained band. Sequencing chromatograms showed that this amount corresponded to the amino acid yields found during Edman degradation

Analysis of the Collagen-Like Protein in High Cell Density Fed-Batch Fermentations

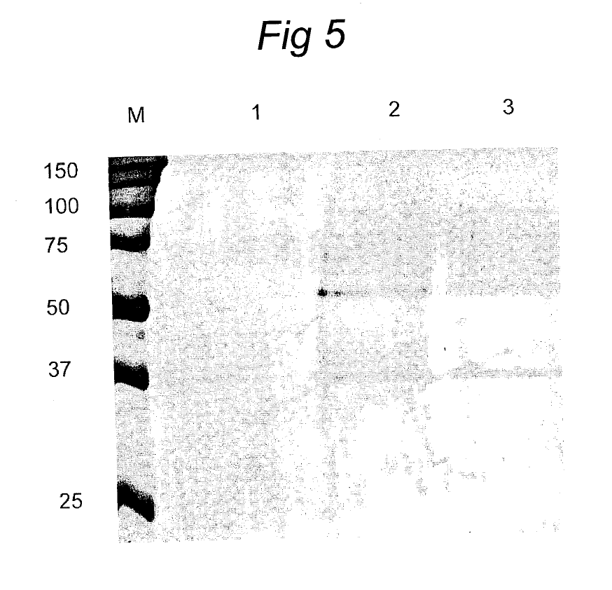

A 38 kD protein could not only be isolated from H. polymorpha shake flasks cultures (as described above), but could also be found in the extracellular medium of high cell density fed-batch cultures supplemented with peptone. Samples of fermentation broth were taken during the fermentation and analyzed by SDS-PAGE after removal of the cells by centrifugation and microfiltration (FIG. 5).

The 38 kD protein is present at a concentration of about 50 mg/L at the end of the fermentation, as estimated from the intensity of the Coomassie-stained band. To verify that this protein was identical to that isolated from shake flask cultures, the N-terminal amino acid sequence was determined (Table 3; see also table 1). Indeed, this appeared to be the case.

TABLE 3
N-terminal amino acid sequences of endogenous H.
polymorpha 38 kDa protein band (see FIG. 5)
isolated from the extracellular medium during
glucose fed-batch fermentation in different media.

| Protein | medium | N-terminal amino | |
|---|---|---|---|
| size (kD) | fed-batch | acid sequence | |
| 38 | CSD | GPPGPPGPPG | (SEQ ID NO:17) |
| 38 | PSD | GPZGPZGPZG | (SEQ ID NO:18) |

Z = 4-hydroxyproline, CSD = casein hydrolysate supplemented dextrose medium, PSD = peptone supplemented dextrose medium

However, it was now not hydroxylated. The amino acid analysis of total extracellular protein present at the end of the fermentation showed high glycine and proline content (18 and 10 mol %, respectively), indicating a significant overall contribution from collagenous protein domains. Hydroxyproline was indeed not present in amino acid analysis.

In contrast to shake flask cultures, heat treatment is apparently not necessary to isolate the collagen-like protein from the cells grown in the fermenter. Possibly, the protein is mechanically released from the cells by shearing forces due to agitation in the fermenter.

When fermentation basal salt medium was supplemented with peptone instead of caseine hydrolysate in a fed-batch fermentation experiment, again the 38 kD protein was found in the medium. N-terminal sequencing revealed the same primary amino acid sequence of several successive [Gly-Pro-Pro] triplets, but the prolines in the most C-terminal position of the triplets were now hydroxylated to 4-hydroxyproline (Table 3). Since the same 38 kD protein with multiple N-terminal [Gly-Pro-Pro] triplets was found, irrespective of the presence of peptone, the possibility that this protein was derived from the peptone can be excluded.

Comparable experiments as presented in table 1 to investigate the specificity of the induction for the supplement added to the growth medium were performed for the endogenous H. polymorpha gelatine production. Analysis of the 38 kDa band showed identical results as obtained for the production of the 15 kDa recombinant band. Only in the presence of peptone and the <10 kDa peptone fraction hydroxyproline residues were observed, irrespective of constitutive or MeOH induced expression. Addition of casamino acids, free 4-hydroxyproline or free aminoacids to the growth medium did not result in the formation of hydoxyproline residues.

Because 4-hydroxyproline occurs exclusively in the most C-terminal position of the [Gly-Pro-Hyp] triplets in the 38 kD protein, it is highly unlikely that free 4-hydroxyproline, derived from fully degraded peptone in the culture medium, is incorporated into the protein during protein synthesis. Such specificity would either require the existence of 4-hydroxyproline-specific codon(s) and tRNA different from those of proline, or else require ribosome-mediated recognition of the sequence context of the collagen-encoding mRNA or the newly synthesized, unfinished protein stretch. Indeed control experiments showed that incorporation of free hydroxyproline is not the cause of the occurrence of hydroxyproline in the collagen product. Thus, in contrast to earlier reports for Pichia pastoris (Vuorela et al. 1997) and Saccharomyces cerevisiae (Vaughan et al. 1998), it can be concluded that H. polymorpha contains an endogenous prolyl 4-hydroxylase, which hydroxylates in a site-specific manner the proline in the Yaa position of the [Gly-Xaa-Yaa] sequence to 4-hydroxyproline.

The endogenous enzyme of H. polymorpha may be used for the hydroxylation of recombinant proteins expressed in this organism, or else, the enzyme may be expressed as a recombinant enzyme in a heterologous host, for hydroxylation of various recombinant protein substrates in such a host.

1. Method for the production of collagen-like compounds containing hydroxylated proline residues characterised by using a fungal prolyl hydroxylase.

2. Method according to claim 1 in which the prolyl hydroxylase is from a uni-cellular fungus, preferably from Hansenula polymorpha.

3. Method according to claim 1 or 2 in which the collagen-like compounds are produced in a fungus or fungus-like eukaryotic microorganism.

4. Method according to claim 3 in which the fungus is a uni-cellular fungus.

5. Method according to claim 3 or 4 in which the collagen like compounds are produced in Hansenula polymorpha, Pichia pastoris, Saccaromyces cerevisiae, Kluyveromyces lactis, Yarrowia lypolitica or Cryptococcus curvatus, preferably in Hansenula polymorpha.

6. Method according to any of the preceding claims in which recombinant collagen-like compounds are produced.

7. Method according to claim 6 in which the recombinant collagen-like compounds are exogenous to the host organism.

8. Method according to claim 6 in which the recombinant collagen-like compounds are over-expressed endogenous collagen-like compounds to the host organism.

9. Method for the production of endogenous fungal collagen-like compounds comprising the steps of culturing a fungus or fungus-like eukaryotic microorganism, and isolating the endogenous fungal collagen-like compound.

10. Method according to claim 9 in which the endogenous fungal collagen-like compound is from Hansenula polymorpha.

11. Method according to claim 9 or 10 in which the fungus is a uni-cellular fungus, preferably Hansenula polymorpha.

12. Method according to any of the preceding claims in which the hydroxylation of proline residues by the fungal prolyl hydroxylase is controlled by the addition of a collagen-like oligopeptide.

13. Method according to claim 12 in which the collagen-like oligopeptide is gelatine hydrolysate selected from gelatone and peptone.

14. Method according to claim 12 or 13 in which the collagen-like oligopeptide has an isoelectric point of higher than 7.

15. Method according to any claims 1-11 in which the hydroxylation of proline residues by the fungal prolyl hydroxylase is controlled by the addition of an extensin.

16. Collagen-like compound obtainable according to any of the preceding claims.

17. Composition comprising a prolyl hydroxylase from a fungus, preferably from Hansenula polymorpha.

18. Prolyl hydroxylase from a fungus, preferably from Hansenula polymorpha.


SEQUENCE LISTING (18 sequences)

SEQ ID NO: 1 - DNA, 27 bp, artificial sequence
  Primer for PCR 1A1-1FW
cttcccagatgtcctatggctatgatg

SEQ ID NO: 2 - DNA, 23 bp, Artificial sequence
  Primer for PCR AMO
tgtccttggtctccttgtgcacg

SEQ ID NO: 3 - protein, 9 aa, Hansenula polymorpha
GFQGPPGEP

SEQ ID NO: 4 - protein, 9 aa, Hansenula polymorpha
GFQGPPGEP

SEQ ID NO: 5 - protein, 9 aa, Hansenula polymorpha
  MISC_FEATURE (6)..(6): X in position 6 is 4-hydroxyproline
GFQGPXGEX

SEQ ID NO: 6 - protein, 9 aa, Hansenula polymorpha
GFQGPPGEP

SEQ ID NO: 7 - protein, 9 aa, Hansenula polymorpha
GFQGPPGEP

SEQ ID NO: 8 - protein, 9 aa, Hansenula polymorpha
  MISC_FEATURE (6)..(6): X in position 6 is 4-hydroxyproline
GFQGPXGEX

SEQ ID NO: 9 - protein, 9 aa, Hansenula polymorpha
  MISC_FEATURE (6)..(6): X in position 6 is 4-hydroxyproline
GFQGPXGEX

SEQ ID NO: 10 - protein, 9 aa, Hansenula polymorpha
GFQGPPGEP

SEQ ID NO: 11 - protein, 9 aa, Hansenula polymorpha
GFQGPPGEP

SEQ ID NO: 12 - protein, 9 aa, Hansenula polymorpha
  MISC_FEATURE (6)..(6): X in position 6 is 4-hydroxyproline
GFQGPXGEX

SEQ ID NO: 13 - protein, 9 aa, Hansenula polymorpha
GFQGPPGEP

SEQ ID NO: 14 - protein, 9 aa, Hansenula polymorpha
GFQGPPGEP

SEQ ID NO: 15 - protein, 20 aa, Hansenula polymorpha
  MISC_FEATURE (17)..(17): X in position 17 is un-identified
EASLGFDLGVQATDGSXKTA

SEQ ID NO: 16 - protein, 21 aa, Hansenula polymorpha
  MISC_FEATURE (3)..(3): X in position 3 is 4-hydroxyproline
GPXGPXGPXGPXGPXGPXGPX

SEQ ID NO: 17 - protein, 10 aa, Hansenula polymorpha
GPPGPPGPPG

SEQ ID NO: 18 - protein, 10 aa, Hansenula polymorpha
  MISC_FEATURE (3)..(3): X in position 3 is 4-hydroxyproline
GPXGPXGPXG